(19) United States
(12) Patent Application Publication
(10) Pub. No.: US 2017/0281394 A1
(43) Pub. Date: Oct. 5, 2017

(54) METHOD AND DEVICE FOR NASAL DILATION BY APPLYING FORCE TO A TARGET CHEEK AREA WITHOUT MANDIBULAR DISPLACEMENT

(71) Applicant: James P. Viken, Eden Prairie, MN (US)

(72) Inventor: James P. Viken, Eden Prairie, MN (US)

(21) Appl. No.: 15/631,632

(22) Filed: Jun. 23, 2017

Related U.S. Application Data
(63) Continuation-in-part of application No. 14/070,910, filed on Nov. 4, 2013.
Application No. 14/070,910 is a continuation of application No. 12/945,826, filed on Nov. 12, 2010, now abandoned.
(60) Provisional application No. 61/260,682, filed on Nov. 12, 2009.

(51) Int. Cl. A61F 5/08

(52) U.S. Cl. CPC A61F 5/08

(57) ABSTRACT

A nasal dilation device and method of use for depressing targeted areas of cheek tissue providing for enhanced inhalation and exhalation via dilation of nasal passageways away from the targeted areas. The device may be used to enhance respiration during a variety of activities, and is especially beneficial during the use of positive airway pressure (PAP) type medical devices. The device may include pads adapted to engage target areas of a user's cheek and to maintain cheek tissue against a bone surface during inhalation and exhalation. The device includes a support element coupled to the pads to maintain the pads at the targeted areas during use.

### RELATED APPLICATIONS

This application claims the benefit of U.S. application Ser. No. 14/070,910, filed Nov. 4, 2013, which claimed the benefit of U.S. application Ser. No. 12/945,826, filed Nov. 12, 2010, which claimed the benefit of U.S. Provisional Application No. 61/260,682, filed Nov. 12, 2009, and each application being incorporated herein by reference.

### FIELD OF THE INVENTION

This invention generally relates to a user interface device and more particularly to a device for enhancing respiration during various user activities via nasal passage dilation.

### BACKGROUND OF THE INVENTION

The prior art is replete with efforts to control snoring and sleep apnea. Among the less intrusive methods of preventing snoring are special pillows, nasal strips, wrist alarms and chemical sprays. Shaped pillows have been designed to realign the back and spine and to relieve muscle stress during sleep, while the adhesive nasal strips hold open the nostrils of a sleeper to improve breathing. Wrist alarms are designed to detect the noise of snoring and to partially arouse a sleeper, ideally to prompt the person to move to a less troublesome position. Chemical sprays contain oils and glycerin that can coat the mucus membranes of the pharyngeal passageway and decrease the noise associated with snoring. For mild cases, these remedies may be all that is necessary, or relief may be found by reducing triggering factors, i.e., losing weight, stopping smoking, and/or decreasing alcohol consumption. However, for more severe cases, the easy solutions usually do not solve the problem. The user can shift and move off of a pillow, while the nasal strips will not help an obstruction in the pharynx. A wrist alarm does not allow the wearer to get a good night's sleep and chemical sprays can wear off in a few hours. For more serious cases, and especially where sleep apnea is involved, the options have included Positive Airway Pressure (PAP), mouth guards that reposition the lower jaw, and surgery.

Motion of the cheeks inward and outward can occur during certain physical exercise, during use of a positive air pressure (PAP) type device, or during the playing of an exhalation-powered musical instrument. This cheek motion can create a variability of the inner volume of the mouth cavity and cause an air accumulator effect to occur, thus making inhalation and/or exhalation less direct and efficient. This air accumulator effect can make the automatic controls of a PAP type device less sensitive, and can result in the use of higher air delivery pressure than would otherwise occur. Unnecessarily higher air delivery pressures in a PAP type device can make the use of a PAP type device less comfortable and decrease patient compliance with the prescribed treatment. Cheeks expanded by air pressure inside the oral cavity and significant cheek motion can be judged unpleasant by PAP type device users. Therefore a need exists to limit significant cheek motion during use of a PAP type device or during certain physical activity.

Obstructive Sleep Apnea (OSA) and other sleep disorders can contribute to the onset of other serious chronic health problems such as high blood pressure, obesity, heart disorders, diminished concentration, and excessive tiredness during the day, falling asleep while driving, and depression and anxiety disorders. Untreated OSA and other sleep disorders may contribute to serious health disorders such as autoimmune disorders and even cancer. OSA and other sleep disorders which are untreated or inadequately treated can reduce one's quality of life and even seriously damage one's health over time and may contribute to a significantly shorter life expectancy.

### SUMMARY OF THE INVENTION

Nasal dilation devices of the present invention engage targeted regions away from a user's nose. An inwardly directed force is applied at the targeted regions to limit significant cheek motion during inhalation and exhalation and with substantially no external forces being directly applied to the user's zygomatic bone or mandible by the nasal dilation device. The user's mandible is maintained in its normal resting position while nasal passageways are dilated. The target area is located between the mandible and zygomatic bone of the user. Rather counterintuitively, an inwardly directed force applied at a target area between the user's zygomatic bone and mandible results in dilation of nasal passageways away from the target area.

The nasal dilation device enhances respiration during exercise or pursuit of individual or team sports, and enhances respiration while at rest or anytime and during any leisure activity. The nasal dilation device's specific size, shape, including shape of its inner cheek facing surface, and the force used to press it against each cheek can be customized to the individual user and the specific application. The nasal dilation device can be used in combination with PAP type devices and other applications where it is helpful to enhance respiration by preventing significant cheek motion which can occur during any exercise, including sport activities and playing of musical instruments.

A pad of the nasal dilation device is pressed and held in place against the cheek at the target area with sufficient force to move the cheek inward to make contact with the teeth, gums and or upper or lower jaw, e.g., maxilla surfaces, but not so much force to abrade, bruise or harm the teeth, gums or upper or lower jaw or the jaw joint. The use of the nasal dilation device can prove useful by enhancing inhalation or exhalation with any activity which can produce significant cheek motion, including the forceful playing of exhalation powered musical instruments where strength of breath and stamina are important considerations. When pressed against each cheek with adequate force necessary to limit significant cheek motion, the nasal dilation device may make respiration more efficient during physical exercise, especially strenuous physical exercise, and enhances respiration any time, including during non-exercise.

A nasal dilation device embodiment of the present invention offers a means of preventing significant cheek motion during inhalation or exhalation which reduces an air accumulator effect of the mouth cavity. Embodiments of the invention can be used to enhance respiration in many diverse applications, including while recreating or while using a PAP type device. Other useful applications in medicine, leisure and industry will become clear as these devices gain more use.

In one embodiment, a nasal dilation device engages a target area of the cheek in order to enhance inhalation or exhalation by preventing significant cheek motion and dilating nasal passageways. In order for the device to be maximally effective it should be positioned to depress the targeted area of the cheek toward the inside of the mouth to cause the cheek to make contact with the teeth and gums. The device should be large enough and have its inner cheek side configured in a curved or slope manner such that it is able to provide the necessary contact with the cheek in order to prevent significant cheek motion during inhalation or exhalation.

The nasal dilation device can be used to enhance respiration, including, but not limited to, during the use of medical equipment such as positive airway pressure (PAP) type devices, or during the pursuit of sports. It can also be used to assist in the playing of exhalation powered musical instruments. The invention can be used to increase the effectiveness of the medical use of PAP type medical devices, including those which are non-automatic such as a basic CPAP device, as well as those that are automatically self adjusting such as an automatic CPAP, BiPAP, Bi-Level, V-Pap, C-Flex, A-Flex or the like which are typically used to treat Obstructive Sleep Apnea (OSA) and other sleep disorders, and which automatically adjust their air delivery pressure based on the user's response, as indicated by sensors and processed by complex algorithms and microprocessors.

One object of this invention is to increase user comfort as a means of increasing user compliance when used with a PAP medical device. It is well known in the art that compliance with a PAP type device is reduced by a user's discomfort while using that PAP type device. Therefore establishing and maintaining maximum comfort during the use of the PAP type device is an important and key factor in determining compliance. Any significant motion of the cheeks during use of a PAP type device can be perceived by some users as uncomfortable when it becomes noticeable and this is a common occurrence if higher air delivery pressures are used, or such higher air pressures occur as a response of any automatic, self-adjusting controls of any PAP type device.

Unrestricted cheek movement during use of a PAP type device can create a hydraulic accumulator effect which can reduce the effectiveness of the pressure transmission of the PAP device resulting in delayed pressure transmission. This delay of pressure transmission to the airway can necessitate higher PAP pressure settings than would otherwise be required to maintain adequate ventilation of the lungs. The accumulator effect can therefore increase the effort of the lungs needed to produce exhalation. Therefore, use of the nasal dilation device can decrease the effort of the lungs to produce inhalation and exhalation and may allow the use of lower settings of the PAP type device, enhancing comfort and compliance with the prescribed treatment.

Significant cheek motion during use of a PAP device itself is judged uncomfortable in some patients, and preventing cheek motion can contribute to patient comfort during their use of a PAP type device. Use of the nasal dilation device to depress the approximate center of the cheek can decrease the effort required to exhale by significantly preventing the accumulator effect that is associated with significant cheek motion, and can thereby increase the comfort of the patient while helping to elicit a quicker more direct response in any automatic, self-adjusting controls that the PAP type device may have, helping to keep the pressure of the air delivered by the PAP type device to a minimum level by reducing the amount of OSA or sleep disorder events.

An important object of the nasal dilation device is to increase the responsiveness of the automatic self-adjusting PAP device's control systems to allow a lower air delivery pressure setting than otherwise might be possible. This can help to increase patient comfort if the pressure can be somewhat reduced while still being adequate to prevent the occurrence of OSA or other sleep disorder events. It is known in the art that the higher the air delivery pressure by the PAP device, the greater the tendency for air to leak around the mask, whether it be a nose only mask, a full face mask or a hybrid form of the two. When air leaks occur due to higher air delivery pressures being used, then the solution typically is the tightening of the straps or headgear used to hold the mask type device securely to the face. It is known that tightening of the straps can decrease patient comfort in some cases.

During the use of a PAP type device, holding the air delivery pressure down to the lowest possible level while still high enough to prevent the occurrence of OSA is an important consideration for PAP and mask device designers. Air leaks through the mask device, which typically occur at the edge seals of the full face type masks, can generate annoying noise which can wake the patient or his or her sleeping partner. When using an automatic self-adjusting PAP type device, these air leaks through the edge seals of the mask will typically cause the device to automatically self-adjust to a higher air delivery pressure output to compensate for the leakage and pressure reduction. The use of the nasal dilation device can result in a barrier provided against the side seals of the full face mask and can help prevent these unwanted air leaks which can be annoying and cause an unwanted increase in the pressure output of the PAP type device.

Therefore, another important object of the use of this invention is to reduce the air leakage of the side seals of a full face mask during the use of a PAP type device by arranging or positioning the invention to make contact with or to reinforce those seals, thereby helping to keep those seals snug against the front sides of the cheeks, and/or serving as a barrier to prevent those seals from being displaced outward and producing unwanted leakage of air from the mask.

Another object of the nasal dilation device is to provide an inexpensive, easy to manufacture means to prevent significant cheek motion in many different activities including but not limited to recreation or during the use of a PAP type devices.

Another object of the invention is to provide a relatively inexpensive, easy to manufacture, easy to wear and use device which can be made integral to a PAP type device mask, chin strap or headgear, or easily installed on a headgear or chin strap for use with a PAP type device, or which can be made to be a free standing support structure similar to a pair of ear phones but positioned at the approximate center of the cheeks for many different uses, thereby providing a useful invention in many different forms which are simple and cheap to manufacture, thereby contributing to less expensive manufacturing in order to reduce carbon emissions that would otherwise be higher.

Another object of the present invention is a nasal dilation that depresses the cheek at the target area to minimize or prevent an air accumulator effect within the oral pharygeal cavity in order to enhance breathing without abrading, bruising or harming the inner cheek, gums, or teeth, especially when using a PAP type unit or engaging in activities which involve hard exhalation, thereby keeping the volume of the oral pharyngeal cavity minimal during respiration, especially during exhalation which would otherwise displace the cheeks outward and expand the oral pharygeal cavity.

Other purposes will appear in the ensuing specification, drawings and claims. The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 is a side elevational view of an embodiment of a nasal dilation device of the present invention.

FIG. 2 illustrates a target area for positioning of the device of FIG. 1

FIG. 3 is a perspective view of the device of FIG. 1.

FIG. 4 is a portion of the device of FIG. 1.

FIG. 5 is a side elevational view of a second embodiment of a device of the present invention.

FIG. 6 is a side elevational view of a spring and pad portion of the embodiment of FIG. 5.

FIG. 7 is a side elevational view of a third embodiment of a device.

FIG. 8 is a spring and pad portion of the embodiment of FIG. 7.

FIG. 9 illustrates a side elevational view of a fourth embodiment of a nasal dilation device of the present invention.

FIG. 10 is a side elevational view of a fifth embodiment of a nasal dilation device of the present invention.

FIG. 11 is a spring and pad portion of the embodiment of FIG. 10.

### DETAILED DESCRIPTION OF THE INVENTION

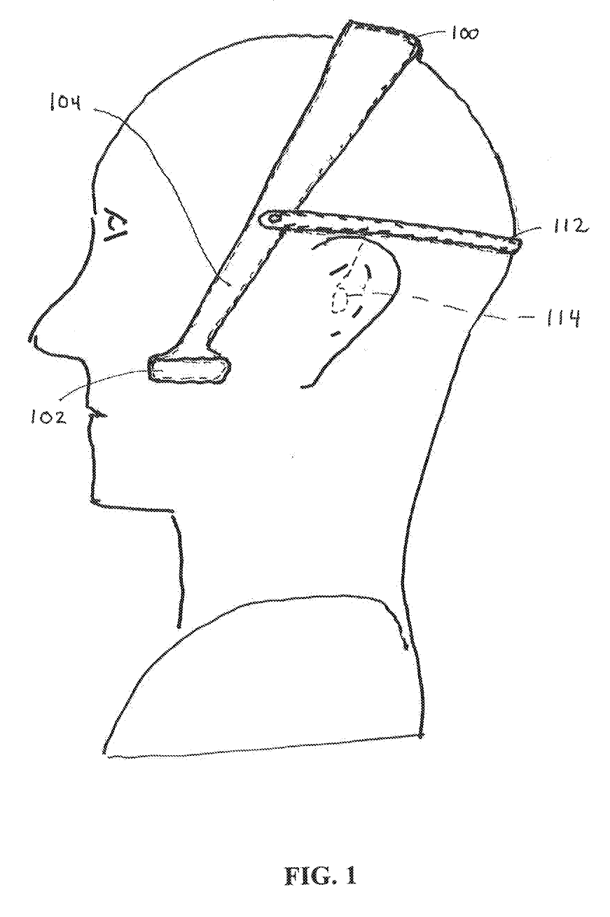

FIG. 1 depicts another embodiment of the invention in a free standing, yoke supported form, identified as nasal dilation device 100. Nasal dilation device 100 is a free standing unit including a spring yoke 104 for support and placement of a pair of affixed pads 102.

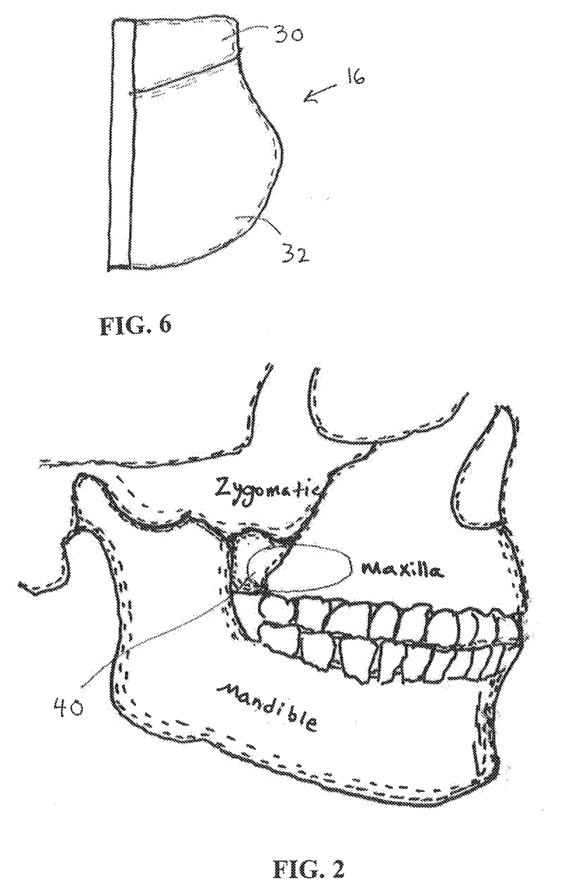

Referring to FIG. 2, the target area is defined as region 40, generally that part of the cheek where the lower lateral pterygoid process bone intersects with a maxilla bone and a zygomatic bone (also called the zygomatic arch or cheek bone). The target area 40 is preferably centered between the zygomatic bone and the upper teeth. The infratemporal surface of the maxilla is contained in the target area 40. The posterior superior alveolar nerves cross through the target area 40. The nasal dilation devices of the present invention preferably provide a force of between about 0.5 lbs-f to 3 lbs-f to the target areas 40 on each side of the user's head. Forces in the range of about 0.05-1.5 lbs-f are delivered to the target areas 40 via various pads of nasal dilation devices of the present invention. These external forces applied to the target area 40 collapse the check tissue against the teeth surfaces of the user and counterintuitively dilate nasal passageways away from the target area 40. Substantially no external force is directly applied by the nasal dilation device to the zygomatic bone or mandible of the user. Minimal inwardly directed forces may be applied against the lower teeth, however, substantially no external force is applied toward moving the mandible forward or rearward. The nasal dilation assembly 100 provides a force at the target area 40 to dilate nasal passageways while the mandible remains in its normal resting position.

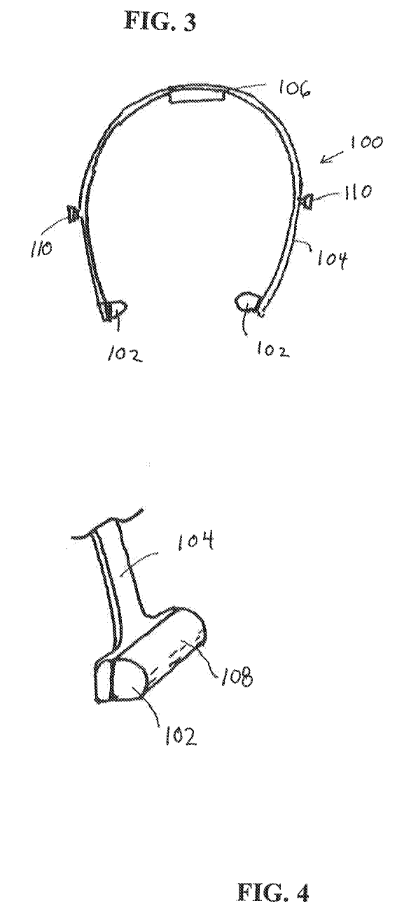

As shown in FIG. 3, a top pad 106 is affixed to spring yoke 104 for user comfort and positioning. Pad 106 is compressible with rebound memory such that it can provide and apply upward force to spring yoke 100. Different thicknesses of pad 106 and different rates of compressibility can be used in order to provide different levels of pressure to the target area 40 while nasal dilation assembly 100 is properly arranged and positioned on a user.

As shown in FIG. 4, pad 102 has a pad surface 108 which is convex. Double sided sticky tape (not shown) can be applied to the convex pad surface 108 to help keep pad 102 in proper position while arranged and positioned on the head of a user. This tape is available as a medical grade tape and has uses in affixing medical and hair appliances. Alternatively medical grade glue can be applied to the convex surface of pad surface 108. In another embodiment, pad 102 can be manufactured with a surface comprised of a plastic or rubber like material which remains sticky but will not erode or transfer to the user, even after washing or using. These types of materials are available from several different manufacturers and can be used in the manufacture of pads like pad 102 to help keep nasal dilation assembly 100 in proper position during use, especially during hard physical exercise with a lot of physical movement and/or jarring.

Spring yoke 104 can be made in several different sizes such as small, medium, large, and extra large to accommodate different users with different sized heads, or can have telescoping adjustment means built into the lower ends of each side or anywhere on each side or at the top (not shown). Spring yoke 104 is provided with a knob 110 on each side, and the knobs 110 are for retaining a strap 112, which is shown in FIG. 1. Strap 112 is somewhat elastic and may be made of a rayon type strapping which has some rubber woven into it for mild elasticity, however many other choices are available for use in constructing strap 112 which would be suitable. Strap 112 is provided with an orifice at each end which is insertable over knob 110 to provide for retention of strap 112 in proper position when nasal dilation assembly 100 is arranged and positioned properly on the user as shown in FIG. 1.

Nasal dilation assembly 100 is arranged and positioned on the head of a user as shown in FIG. 1, and with each of pads 102 arranged and positioned to make contact with the target area of the cheek of the user. Counterintuitively, application of an inwardly directed force at the target area 40 results in dilation of nasal passageways away from the target area 40.

Many different configurations for spring yoke 104 can be utilized, some of which include a spring yoke designed to fit from under the chin or to fit from behind the head. Alternatively a yoke can be integrated into a headphone unit or into almost any helmet design used in sports if such design takes into account safety needs and constructs the pair of pads 102 to be suitably collapsible or displaceable upon hard impact, especially for high contact sports. Nasal dilation assembly 100 can be designed and constructed such that pad 102 can be made to be adjustable at the bottom ends of spring yoke 104, with such adjustment for angle to spring yoke 104, or to the target area, without departing from this novel art. Manufacturing nasal dilation assembly 100 with said adjustments can allow the user to specifically adjust each pad for the desired and best contact and pressure to be applied to the target area.

Nasal dilation assembly 100 can be manufactured in many different configurations to fit the personal preference of different individual users. It can have a spring yoke configured and constructed which will allow it to be placed under the chin, or around the neck in back, or even placed in front of the head, above or below the nose. It can be constructed to contain ear phones for audio, enabling the user to listen to a radio, iPod or cell phone while using the nasal dilation device 100. In addition, a yoke can be constructed of a rigid material which holds its approximate form even when worn, and the spring action for asserting pressure against the target area can be supplied by the compressibility of the pad such as pad 102 or a spring system inside or behind it. The pad can be constructed in a larger combination form such as shown in FIG. 15, which will depress the approximate center of the cheek and the cheek area just below the zygomatic bone.

FIG. 1 shows strap 112 arranged and positioned to hold nasal dilation assembly 100 in proper position. The use of pad 106 (shown in FIG. 3) to apply pressure between the user and spring yoke 104, along with strap 112 and the pressure applied bilaterally to each cheek target area by the pair of pads 102, provides sufficient support to keep nasal dilation assembly 100 in position in most situations, including vigorous exercise. An additional strap (not shown) could be affixed to the pair of knobs 110 and arranged and positioned to cross the forehead of the user or even under the nose of the user in order to provide good support.

Of course, if the pair of pads 102 is provided with double sided medical grade adhesive or tape, then even more stability can be provided to nasal dilation assembly 100. If the nasal dilation assembly 100 is carefully fit with suitable pressure applied at pad 106 and suitable pressure applied at the bilateral pair of target areas of the cheeks of the user, then it is possible to construct nasal dilation assembly 100 without use of any strap(s) such as strap 112. Pad 102 itself can be constructed of a compressible foam plastic or rubber like material with rebound memory that can be arranged to exert sufficient and desired depression of the target area if a rigid yoke structure is utilized to provide support for the pair of pads used such as pad 102.

Spring yoke 104 can be shaped to provide some upward force from the horizontal to the target area, less than that applied horizontally to the target area. In this embodiment an upward angle of about 35 degrees from the horizontal was used to vector the upward force into the target area. Other angles could be used including up to a vertical force, and this capability could be manufactured into the nasal dilation assembly 100 allowing easy adjustment by the user. It was determined by the inventor during experimentation that nasal dilation assembly 100 works somewhat more effectively in some individuals when there is some upward angular force applied to the target area to press snugly against the lower end of the zygomatic bone and the lower side of the lower lateral pterygoid process bone while exerting slight inward pressure against the upper portion of the maxilla bone. These pressures should be sufficient to depress the target area of the cheek suitably, and also for optimal effectiveness can have some of the force applied at an upward inward angle from the horizontal, which can provide more enhancement of breathing in some, all without abrading, bruising or harming the inner side of the cheek, the gums or the teeth.

Spring yoke 104 is strong enough to assert adequate pressure to each of the bilaterally positioned pads 102 to keep the target area suitably and adequately depressed without abrading, bruising or harming the array of upper teeth, gums or inner cheek. In one embodiment, a force of approximately 1.20 lbs was applied through each pad 102. However individual needs can vary and adjustments can be designed, manufactured and incorporated into the finished product which will allow pressure to be adjusted to an increase or to a decrease within a fairly broad range, generally within 0.5 lb to 3 lbs, but individual cases could require less or more. Pad 106 can also be supplied in different thicknesses and hardnesses, if suitable means for adjusting length are not provided to nasal dilation assembly 100. In addition telescoping means can be provided to the top of spring yoke 104 to increase or decrease the pressure applied to each of the pair of pads 102.

Nasal dilation assembly 100 is designed as a freestanding unit which can be used alone or with many mask assemblies for PAP usage. Nasal dilation assembly 100 can be used alone to enhance breathing and respiration during exercise or sports, or anytime based on the personal preference of the user. It may also be helpful for some who play exhalation powered musical instruments by enhancing exhalation when it is used with the small pad shown in FIG. 1, which depresses the area of the cheek under the zygomatic bone.

When nasal dilation assembly is properly arranged and positioned such that the pair of pads 102 are suitably depressing the target area bilaterally on the cheeks and strap 112 is properly arranged and positioned to retain nasal dilation assembly 100 in proper position, breathing and respiration can be enhanced. The suitable and effective depression of the target area of the user's cheek enhances respiration in a significant number of individuals perhaps by neurological or neuromuscular stimulation means. FIG. 1 shows how nasal dilation assembly 100 appears when properly arranged and positioned whereby the pair of pads 102 are positioned to suitably depress the target area of the cheek. For maximum effectiveness in enhancing breathing and respiration, pressure must be applied to the target area in a sustained and focused fashion, but even minimal pressures appear to enhance breathing and respiration somewhat in a significant number of individuals.

A sustained and significant pressure to the target area appears to directly enhance inhalation as well as exhalation somewhat, even without depressing the approximate center of the cheek. And in addition, it was determined that if some pressure was applied to the target area in an upward angle from the horizontal, there was an additional enhancement of breathing in some individuals. Nasal dilation assembly 100, when properly arranged and positioned, may enhance breathing and respiration during normal sleeping in some individuals, even those without Obstructive Sleep Apnea (OSA) or any diagnosable sleep disorder.

Sustained pressure provided to the cheek area right below the zygomatic bone can enhance breathing and respiration in some individuals. Thus the proper arranging and positioning of nasal dilation assembly can enhance breathing and respiration, and when the nasal dilation is applied to the approximate center of the cheek it can significantly reduce or substantially eliminate an air accumulator effect which can reduce the effectiveness of one's usage of a PAP device. It should be noted that sustained pressure to the cheek area just below the zygomatic bone appears to enhance breathing and respiration perhaps by neurological and/or neuromuscular stimulation.

For a significant number of individuals, sustained pressure properly applied to the target area can serve to enhance breathing and respiration as long as the sustained pressure is applied to this area in an amount great enough to depress this area of the cheek deeply but not too great to abrade the inner cheek, or bruise the upper gums or harm the upper array of teeth. The amount of pressure needed to attain this for any given individual can vary and that must be kept in mind, and different thicknesses of pads can be made available if necessary or pads can be provided with different rebound rates.

The suitable depression of the target area by applied, sustained pressure can be attained by various structures represented in the illustrations or their equivalents, or in the structures disclosed but not shown specifically. For a significant number of individuals it is the sustained and focused application of pressure to this area of convergence of the lower lateral pterygoid process bone, the maxilla bone and the zygomatic bone which allows the user to attain the maximum enhancement of breathing whether or not a particular embodiment disclosed, or an equivalent of that, depresses the approximate center of the cheek. For example if a Cheek Depressor is to be used during the use of a PAP type device or while playing an exhalation powered musical instrument, or while engaging in heavy exercise with a lot of movement and high exertion, a significant number of individuals will benefit from using an embodiment that emphasizes and focuses pressure to depress the target area of the cheek in addition to depressing the approximate center of the cheek. And for other situations where exercise is light, or there is no exercise involved, use of the nasal dilation device in a free standing embodiment is an appropriate choice and will enhance breathing for a significant number of individuals. If an individual is engaging in an activity which requires and produces hard exhalation such as running or heavy exertion sports or playing an exhalation powered musical instrument, then it would be a good choice to use a nasal dilation embodiment which depresses the approximate center of the cheek and also focuses specific pressure on the target area.

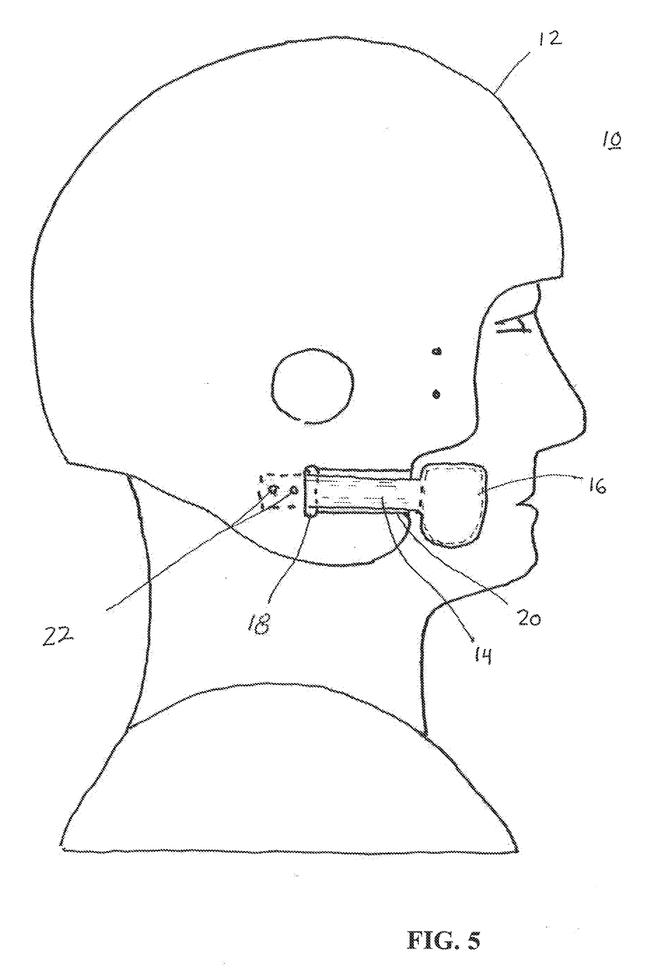

FIG. 5 is a side view illustration of an embodiment of the present invention. A sports helmet assembly 10 includes a shell 12, spring 14 and a check-engaging pad assembly 16 which is attached to spring 14. Helmet assembly 10 includes a padded shell of traditional construction. Spring 14 at one end passes through an aperture 18 of shell 12 and is then fastened to the inside of shell 12. A portion of spring 14 rests in a surface channel 20 in shell 12. As described herein, pad assembly 16 defines a cheek depressing device. Spring 14 is held onto shell 12 via fasteners 22, such as rivets, etc.

FIG. 6 illustrates pad assembly 16 as including two pad portions, a hard pad portion 30 and a soft pad portion 32 which are preferably molded together and affixed to spring 14. Soft pad portion 32 is made of a foam plastic material similar to foam rubber in its properties and is moderately compressible, whereas hard pad portion 30 is generally non-compressible. Soft pad portion 32 is thicker at its approximate center than hard pad portion 30. During use, pad assembly 16 is positioned onto the cheek of the user and is held against the cheek by spring 14. Hard pad portion 30 suitably depresses a target area of the cheek of the user, and soft pad portion 32 suitably depresses the approximate center of the cheek of the user with soft pad portion 32 being compressed by spring 14. Pad assembly 16 can be custom molded, shaped, and constructed specific to each user's cheek anatomy and hard pad portion 30 and soft pad portion 32 can be constructed within pad assembly 16 in a variety of different hardnesses and compressibility to provide for individual differences.

In order to put on or remove football helmet assembly 10, the user must pull springs 14 apart to move pad assembly 16 outward away from shell 12, after which the springs 14 can be slowly released once the helmet is donned. During hard shocks spring 14 will be able to flex somewhat thereby adjusting to the exact temporary position of the users face and head inside football helmet assembly 10. The simultaneous depression of the target area and the approximate center of each cheek can provide enhanced breathing.

A helmet assembly can be constructed with an integral cheek depressor in a variety of alternate ways. For example, a rigid plate which is shaped approximate to the size of the intended cheek depressor can be attached to the lower front edge of each side of the helmet closest to the chin of the user and this plate can be attached by use of a spring hinge which will apply the desired pressure to the specific pad design and size. A smaller plate may be used to hold a pad intended to depress the target area only, or a larger plate may be used to hold a pad intended to depress the target area of the cheek and also the approximate center of the cheek. A combination pad assembly comprised of a hard pad and a soft more compressible pad may also be used.

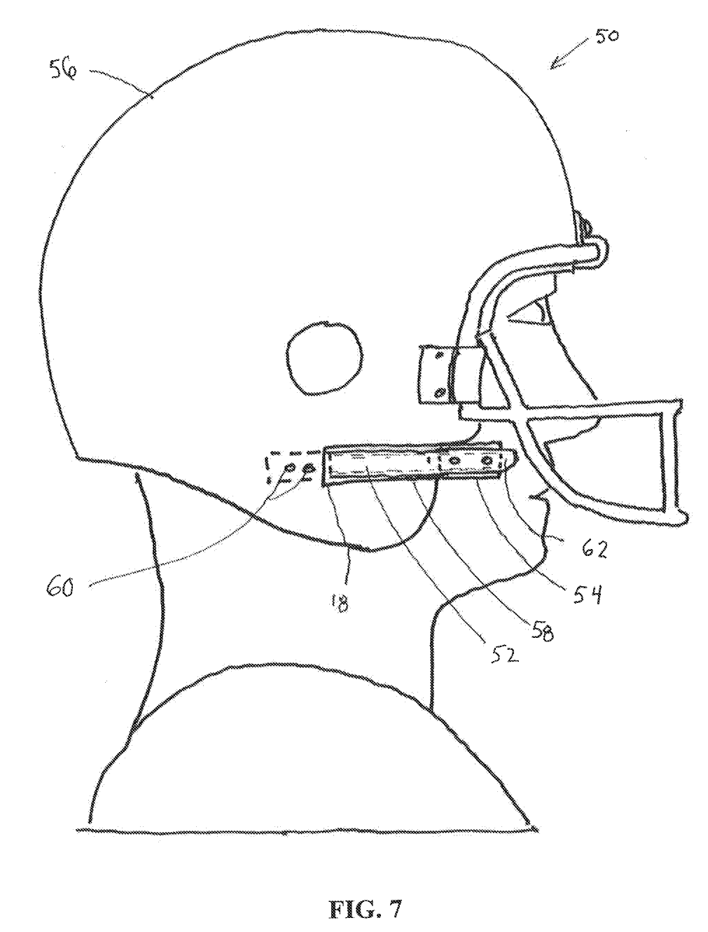

FIG. 7 depicts another embodiment of the present invention. A football helmet assembly 50 includes a bilateral and integral cheek depressor comprised of a spring 52 and pad 54. Football helmet assembly 50 is comprised of a generally rigid shell 56 having a padded interior. Shell 56 is provided an aperture 18 through which spring 52 passes into and through. Shell 56 is also provided with a surface channel 58 deep enough to retain spring 52 at or beneath the outside surface of shell 56 when it is properly installed and in proper operating position after football helmet assembly 50 is in proper wearing position.

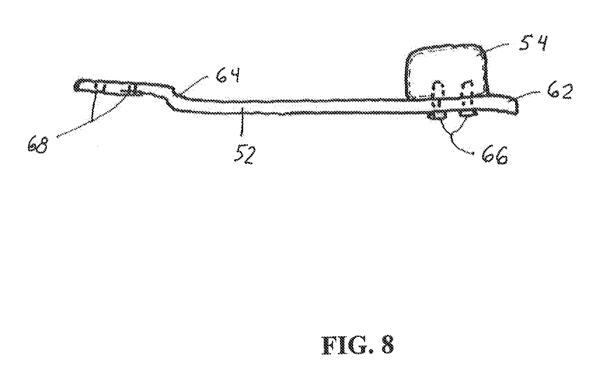

FIG. 8 shows spring 52 as being bent to allow it to be inserted through aperture 18 and to then engage against surface channel 58 after it has been secured in place, via for example a pair of rivets 60. Spring 52 is provided with a tab 62 at one end which allows the user to grasp spring 52 and pull it and its attached pad 54 away from shell 56 in order to either put on or remove football helmet assembly 50.

Spring 52 is provided with a pad 54 which is substantially firm and is molded, such as with a medical grade silicone compound. Pad 54 has a slight convex shape to its inner surface which faces the user. FIG. 8 shows spring 52 to have a double opposing bend 64 provided for spring 52 to fit properly into and through aperture 18. If the user desires a slightly upward force applied to the target area, a slight twist can be added to spring 52. A plurality of fasteners 66 are used to connect spring 52 to pad 54. Spring 52 includes a plurality of apertures 68 through which fasteners 60 are received during installation.

Football helmet assembly 50 of FIG. 7 provides an integral cheek depressor allowing a football player to enhance his breathing while playing football. During hard shocks spring 52 will be able to flex somewhat thereby adjusting to the exact momentary positional change of the user's face and head inside football helmet assembly 50. Some players might prefer a pad 54 that extends downward farther toward the approximate center of the cheek below the target area in addition to a pronounced depression of the target area.

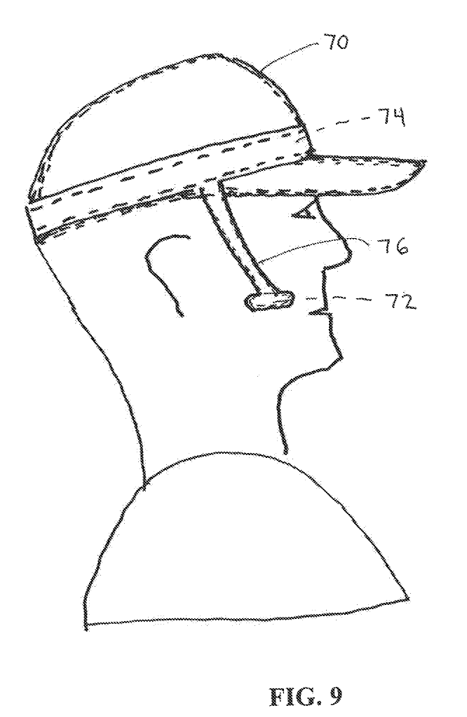

FIG. 9 shows user another embodiment of the present invention including a cap assembly 70 including pads 72 held tightly in place against the target area of the cheek via spring frame 74 and spring leg 76. Cap assembly 70 may be a baseball-style cap.

Spring frame 74 and spring leg 76 in this embodiment are constructed of a spring material, such as stainless steel, which will hold its shape and exert generally constant pressure inwardly thereby directing forces into the target area of the user. Portions of cap assembly 70 may be constructed of fabric, such as a heavy duty cotton/polyester weave. The pair of spring legs 76 have pads 72 affixed to each end. Pads may be constructed of molded medical grade silicone. The inner surface (facing user) of pads 72 is somewhat rounded or convex. Spring leg 76 can be curved inward at its lower section in such a way that pad 72 can be arranged and positioned to provide a somewhat angular upward force against the target area of the cheek in addition to an approximately horizontal force applied inward against the target region. In some users a slight upward angle to the force applied provides somewhat better breathing than a straight horizontally applied force. Double sided medical grade tape tabs can be constructed of the proper size and applied to the cheek side surface of pads 72 to help keep pads 72 in proper position during vigorous exercise.

Stainless steel alloy is a reasonable choice for compactness, durability and rebound memory and is selected for this embodiment, but many other materials could be used to construct spring frame 74 and spring leg 76 including other types of metals, plastics or composites providing higher strength, better rebound memory and light weight. Spring frame 74 is preferably constructed of a spring type material with good rebound memory, but it is also possible to construct frame 74 of a mostly rigid material and provide compressible pads with rebound memory instead. Spring legs 76 may be provided with a telescoping adjustment means.

A cap can also be constructed to include larger cheek depressors targeted to the target area in order to decrease an air accumulator effect which may occur during hard respiration of some when they engage in vigorous exercise involving maximum exertion and hard respiration. For some a larger cheek pad may provide more optimal breathing than a smaller one. In order to provide for a larger pad 72 or increased force for pad 72 against the target area of the user's cheek, a stronger spring leg 76 can be used to provide more spring force inward.

In order to don cap assembly 70, a user spreads apart the spring legs 76, places the cap in proper wearing position, and then releases the pair of spring legs 76, allowing the pair of pads 72 to move inwardly to press snuggly and depress into the target area of each cheek. Legs 76 can be adjusted by increasing or decreasing their length by use of the telescoping feature built into each leg 76 (not shown but understood in the art) if necessary for good contact between the pad 72 and the target area of the cheek. A double sided medical grade tape tab can be applied to the inner surface of each of the pair of pads 72, which in addition to the tension provided by spring leg 76 will help keep pad 72 in proper position during hard or jarring exercise and will help keep the cap in place. The double sided tape tabs are disposable and can be replaced at each use of the hat or as frequently as desired by the user.

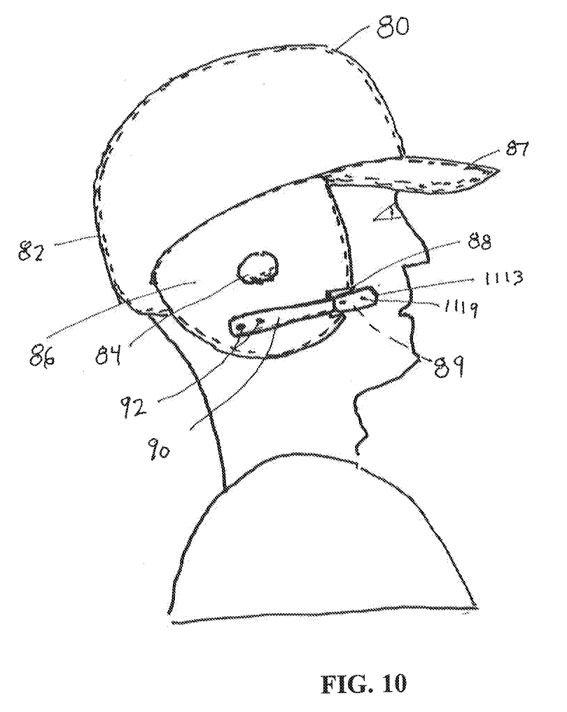

FIG. 10 depicts another embodiment of the invention. A batting helmet assembly 80 is a protective helmet used by baseball players. Batting helmet assembly 80 has a shell 82 which contains an inner padding (not shown) which helps cushion and protect the head from shock when hit by a baseball. An aperture 84 is provided to ear protector cavity 86 to allow the wearer to hear. Batting helmet assembly 80 is typically constructed of a strong resilient type of molded plastic or fiberglass, but carbon fiber can be selected for molding as an even stronger, lighter option. Batting helmet assembly 80 is provided with a visor 87 and a cutaway 88, which is a small cutaway from the bottom front of ear protector cavity 86. Cutaway 88 provides clearance for the penetration of a pad 89 through ear protector cavity 86 to make good contact with the target area of the cheek of the user.

Ear protector cavity 86 is provided with a spring 90 to which pad 89 is provided to its inward facing side toward the user's face. Pad 89 is molded of medical grade silicone which can feel sticky to the touch when cleaned with soapy water. Pad 89 is somewhat convex at its inner side which faces the cheek and has no sharp edges. Spring 90 is comprised in this instance of a high quality stainless steel which provides good strength and rebound memory while providing adequate force against pad 89 to depress the target area of the cheek of the user.

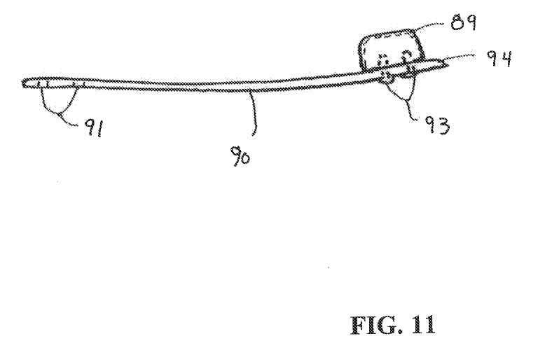

As shown in FIG. 11, spring 90 is provided with apertures 91 at each end. These end apertures 91 are used to attach spring 90 to ear protector cavity 86 via, for example, rivets 92 passing through holes provided to shell 82 (not shown). Threaded fasteners 93 are used to secured pad 89 to spring 90. Other types of fasteners and even glue pads or glue can also be used to affix pad 89 to spring 90. Pad 89 can be provided in different widths in order to provide for individual differences in facial anatomy, thereby insuring proper tension to spring 90 which will in turn provide proper depression of the target area of the cheek. Tab 94 is provided at one end of spring 90 to provide a point for the user to grab and manipulate pad 89 away from the target area of the cheek, so that the helmet can be put on or removed.

As shown in FIG. 10, batting helmet assembly 80 positions pad 89 against the target area 40 of the cheek via spring 90. The integral use of a cheek depressor to a batting helmet assembly 80 allows enhanced breathing for the batter. Batting helmet assembly 80 can be donned or removed by grasping tabs 94 and pulling each spring 90 outward to thereby pull each pad 89 away from the cheek of the user.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A nasal dilation device, comprising:
a pad engaging a target area of the user's cheek and maintaining cheek tissue at the target area against an underlying tooth structure during inhalation and exhalation, the target area being located between above a mandible and below a zygomatic bone of a given human head, and with the pad engaging the target area with a pad force of between 0.5 pounds to 3 pounds to maintain cheek tissue at the target area against the underlying tooth structure, and with substantially no external force being applied to the zygomatic bone or mandible of the user; and
a support adapted to be head worn and providing the pad force at the target area, said pad force being applied by a pair of arms positioned rearward of the pads, with said mandible being maintained in its normal resting position while nasal passageways away from the target area are dilated.

2. The device of claim 1 wherein the head support structure includes is a spring.

3. The device of claim 2 wherein the spring is a yoke structure.

4. The device of claim 1 wherein said pad includes at least two portions having at least two substantially different levels of compressibility.

5. The device of claim 1 further comprising an adhesive for securing the pad in the target area.

6. The device of claim 5 wherein the adhesive is provided by an adhesive tape.

7. The device of claim 1 wherein the pad is a semi-spherical body.

8. The device of claim 1 wherein the pad force is between about 0.5 pounds to 1.0 pounds to the target area.

9. The device of claim 1 wherein the pad is gas-filled.

10. A nasal dilation device comprising:
a pair of pads adapted to be placed against a user's cheeks, with each of said pair of pads being placed at a target area above a jawbone and below a zygomatic bone of the user; and
a head-worn device including a spring structure depressing the pair of pads against said user's cheek tissue, said spring structure providing a cheek tissue-depressing force to said pair of pads resulting in an application of an external force to said user's cheek tissue at the target area and away from both the mandible and the zygomatic bone of the user, said force tending to limit movement of said user's cheek tissue during respiration and maintain inner cheek tissue against the user's gums and teeth during use, and with substantially no external forces being directly applied by the spring structure to either the zygomatic bone or mandible of the user and with substantially no jaw thrusting pressure being applied to the mandible, with said mandible being maintained in its normal resting position while dilating nasal passageways away from the target area.

11. The device of claim 10 wherein said pad includes at least two portions having at least two substantially different levels of compressibility.

12. The device of claim 10 further comprising an adhesive for securing the pad in the target area.

13. The device of claim 10 wherein the pad force is between about 0.5 pounds to 1.0 pounds to the target area.

14. The device of claim 10 wherein the pad is gas-filled.

15. A method using a nasal dilation device of claim 1 comprising:
providing the pad against a user's cheek tissue at a target area between a jawbone and a zygomatic bone of the user;
securing said pad against the user's cheek tissue with the support, said support applying the force through the pad to the user's cheek only at the target area and away from both the jawbone and zygomatic bone of the user; and
dilating nasal passageways away from the target area by restraining movement of the user's cheek tissue by application of said force to said pad.

16. A method of a nasal dilation device of claim 10 comprising:
providing the pair of pads in contact with the target area of the user's cheek tissue located above a jawbone and below a zygomatic bone of the user;
securing the pair of pads in place with the spring structure, with said spring structure providing a force through said pad to maintain contact between the user's inner cheek and teeth and gums only at the targeted area and with substantially no external forces being applied by the spring structure to either the mandible or zygomatic bone of the user; and
dilating nasal passageway by restraining movement of the user's cheek tissue by application of said force at the target area while maintaining the user's mandible in its normal resting position.

17. A method of nasal dilation using a head-worn dilation device, comprising:
engaging a pair of target area of the user's cheek with a pair of pads;
supporting the pair of pads at the target areas with a head worn support including a pair of arms positioned rearward of the pair of pads; and
maintaining cheek tissue at the target areas against underlying tooth structure during inhalation and exhalation, the target area being located above a mandible and below a zygomatic bone of a given human head, with the pair of pads engaging the target areas with a pad force of between 0.5 pounds to 3 pounds to maintain cheek tissue at the target areas against the underlying tooth structure, and with substantially no external force being applied to the zygomatic bone or mandible of the user, and with a user's mandible being maintained in its normal resting position while nasal passageways away from the target areas are dilated by the pair of pads.
